(19) United States
(12) Patent Application Publication
(10) Pub. No.: US 2025/0082569 A1
(43) Pub. Date: Mar. 13, 2025

(54) ORALLY DISINTEGRATING NICOTINE TABLET WITH LOW AMOUNT OF FLAVOR

(71) Applicant: Fertin Pharma A/S, Vejle (DK)

(72) Inventors: Rune Faurby Carlsson, Vejle (DK); Kent Albin Nielsen, Vejle (DK); Rikke Pranger-Rasmussen, Vejle (DK); Bruno Provstgaard Nielsen, Vejle (DK); Helle Martinussen, Vejle (DK)

(73) Assignee: Fertin Pharma A/S, Vejle (DK)

(21) Appl. No.: 18/883,730

(22) Filed: Sep. 12, 2024

(30) Foreign Application Priority Data
Sep. 12, 2023 (DK) PA 2023 70471
Mar. 18, 2024 (WO) PCT/DK2024/050052

(51) Int. Cl. A61K 9/00; A24B 15/16; A61K 9/20; A61K 9/24; A61K 31/465

(52) U.S. Cl. CPC A61K 9/0056; A24B 15/16; A61K 9/2009; A61K 9/2018; A61K 9/2027; A61K 9/2054; A61K 9/209; A61K 31/465

(57) ABSTRACT

An orally disintegrating nicotine tablet for fast nicotine craving relief is disclosed, the tablet comprising nicotine, disintegrant, and at least one sugar alcohol, wherein the tablet comprises less than 0.4% by weight of flavor. A further orally disintegrating nicotine tablet and an oral nicotine tablet is disclosed.

### FIELD OF INVENTION

The invention related to orally disintegrating nicotine tablets according to the claims. Also, the invention relates to an oral nicotine tablet according to the claims.

### BACKGROUND

Nicotine-releasing tablets applied for the purpose of providing a release of nicotine in a user's mouth over a certain period of time is well-known. Much effort has in prior art been put into emulating the nicotine release and oral perception of a cigarette when it is smoked by a user, which means that release profiles from nicotine tablets have been thoroughly investigated in prior art.

It is however an established fact that the one-to-one smoking-emulation is yet to be achieved with other means than a cigarette.

Furthermore, user perception of nicotine tablet may be improved, e.g. since release of nicotine is associated with undesirable irritation in the throat, a side effect known as nicotine burning or simply burning.

An example of an orally disintegrating nicotine tablet is known from PCT application WO 2019/110072 A1, to Fertin Pharma A/S, related to orally disintegrating tablets with disintegration within 60 second from oral administration.

### SUMMARY

The invention relates to an orally disintegrating nicotine tablet for fast nicotine craving relief, the tablet comprising nicotine, disintegrant, and at least one sugar alcohol, wherein the tablet comprises less than 0.4% by weight of flavor.

One advantage of the invention may be that a desirable user experience may be obtained.

Typically, nicotine-containing tablets may comprise flavor in substantial amounts for accommodating taste masking, both in terms of nicotine and also with respect to typically included pH regulating agents, which may have off-note taste. Thus, the flavor induced taste experience may often remain in the mouth for some time after the tablet has been disintegrated. The present inventors unexpectedly found that many users considered this lasting taste to be undesirable and that this could be solved by reducing the amount of flavor below the amounts conventionally used while maintaining an acceptable overall user experience.

Usually, oral release of nicotine may be associated with undesirable taste and mouthfeel, including local irritation of the mouth and throat, also known as nicotine burning. However, in some embodiments of the invention the present inventors unexpectedly found that lowering the amount of flavor in the tablet did not lead to a significant increase in the nicotine burning.

A further advantage of the invention may be that the tablet induces less saliva generation. Without being bound by theory, it is contemplated that less saliva generation facilitates buccal absorption due to increased nicotine residence time and limits swallowing of nicotine. Having a low amount of flavor or even no flavor may thus facilitate decreased salivation as perceived when using the tablet. A decreased salivation may be advantageous for users who e.g. want to discretely use the tablets.

A further advantage of the invention may be that decreased flavor may be desirable for users who uses the tablets by placing the tablet under the lip, i.e. between the gum and the lip. Such use may be advantageous, e.g. with respect to efficient uptake of nicotine and low nicotine burning. Furthermore, by including only the claimed low amounts of flavor or no flavor, local irritation associated with flavor may be minimized or even avoided.

It is further noted that an advantage of the invention may be a combination of fast nicotine delivery, facilitating fast nicotine craving relief, minimized nicotine burning, and/or minimized aftertaste after use of the tablet.

It is noted that in the present context, the term “orally disintegrating nicotine tablet” refers to a nicotine containing orally disintegrating tablet, also referred to as a nicotine ODT, i.e. a tablet that disintegrates relative fast. It is noted that orally disintegrating tablets, or ODTs, may sometimes be referred to as fast disintegrating tablets, or FDTs. In embodiments of the invention, the orally disintegrating nicotine tablet may thus disintegrate within 90 seconds upon oral administration, preferable when administered on the tongue. In embodiments of the invention, the orally disintegrating nicotine tablet may thus disintegrate within 90 seconds upon oral administration, preferable when administered under the tongue (sublingually). In an embodiment of the invention, the orally disintegrating nicotine tablet may thus disintegrate within 5 minutes upon oral administration, such as within 4 minutes upon oral administration, such as within 3 minutes upon oral administration, preferable when administered between the gum and the lip. In embodiments of the invention, the orally disintegrating nicotine tablet may disintegrate within 60 seconds in vitro, preferably when measuring vitro disintegration time in accordance with European Pharmacopoeia 9.0, section 2.9.1, Disintegration of tablets and capsules.

According to an embodiment of the invention, the in vivo disintegration time is measured by at least 6 trained assessors, the trained assessors abstaining from eating and drinking at least 30 minutes before initiation of any test, the tablet is weighted and placed in the mouth, on the tongue, where the in vivo disintegration time is registered as the time point where the tablet disintegration is substantially complete. During the test, the assessors refrained from chewing and did not substantially move the tablet from its initial position. It is noted that whereas the in vivo disintegration time is tested by placing the tablet on the tongue, as described above, the tablet may in some embodiments be intended for use as a sublingual tablet, i.e. below the tongue. Also, in some embodiments, the tablet may be intended to be used between the gum and lip. It is noted that the use of tablets between the gum and lip results in a longer disintegration time.

In an embodiment of the invention, trained assessors, e.g. for in vivo measurement of disintegration time, may be selected as described in the following. The trained assessors are selected based on the following criteria. First, the individuals chewed on a chewing gum base free of buffer for 1 minute, after which the initial pH in a sample from the saliva from each of the individuals is measured with a suitable pH-electrode system, e.g. a stainless steel electrode PHW77-SS. Only individuals having, after chewing on a chewing gum base free of buffer for one minute, an initial pH in the saliva inside the range from 6.7 and 7.3 are selected. These individuals thereby qualify as assessors. One dosage of the tablet is administered on the tongue to at least six assessors. Hereafter, disintegration time is evaluated by the six assessors. Each assessor evaluates each sample twice. Thus, each parameter evaluation is the arithmetic mean of 12 assessments performed on samples from six assessors. During the test, the assessors refrained from chewing and did not substantially move the tablet from its initial position.

According to an advantageous embodiment of the invention, the tablet comprises no more than 0.3% by weight of flavor, such as no more than 0.2% by weight of flavor, such as no more than 0.1% by weight of flavor, such as free of flavor.

In an embodiment of the invention, the tablet comprises 0% to less than 0.4% by weight of flavor, such as 0.01% to 0.3% by weight of flavor, such as 0.05% to 0.2% by weight of flavor, such as 0.05% to 0.1% by weight of flavor, such as free of flavor.

In an embodiment of the invention, one or more flavors are selected from peppermint, menthol, almond, almond amaretto, apple, Bavarian cream, black cherry, black sesame seed, blueberry, brown sugar, bubblegum, butterscotch, cappuccino, caramel, caramel cappuccino, cheesecake (graham crust), cinnamon redhots, cotton candy, circus cotton candy, clove, coconut, coffee, clear coffee, double chocolate, energy cow, graham cracker, grape juice, green apple, Hawaiian punch, honey, Jamaican rum, Kentucky bourbon, kiwi, koolada, lemon, lemon lime, maple syrup, maraschino cherry, marshmallow, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry kiwi, tropical punch, tutti frutti, vanilla, or any combination thereof.

According to an embodiment of the invention, the flavor comprises menthol.

According to an embodiment of the invention, the flavor comprises tobacco flavor.

According to an embodiment of the invention, the flavor consists of menthol and/or tobacco flavor.

According to an embodiment of the invention, the flavor consists of menthol.

According to an embodiment of the invention, the flavor consists of tobacco flavor.

According to an advantageous embodiment of the invention, the tablet comprises liquid flavor.

According to an advantageous embodiment of the invention, the tablet comprises less than 0.4% by weight of liquid flavor, such as no more than 0.3% by weight of liquid flavor, such as no more than 0.2% by weight of liquid flavor, such as no more than 0.1% by weight of liquid flavor, such as free of liquid flavor.

In an embodiment of the invention, the tablet comprises 0% to less than 0.4% by weight of liquid flavor, such as 0.01% to 0.3% by weight of liquid flavor, such as 0.05% to 0.2% by weight of liquid flavor, such as 0.05% to 0.1% by weight of liquid flavor.

According to an advantageous embodiment of the invention, the tablet comprises powdered flavor.

According to an advantageous embodiment of the invention, the tablet comprises less than 0.4% by weight of powdered flavor, such as no more than 0.3% by weight of powdered flavor, such as no more than 0.2% by weight of powdered flavor, such as no more than 0.1% by weight of powdered flavor, such as free of powdered flavor.

In an embodiment of the invention, the tablet comprises 0% to less than 0.4% by weight of powdered flavor, such as 0.01% to 0.3% by weight of powdered flavor, such as 0.05% to 0.2% by weight of powdered flavor, such as 0.05% to 0.1% by weight of powdered flavor.

According to an embodiment of the invention, the tablet comprises powdered flavor and liquid flavor.

According to an advantageous embodiment of the invention, the tablet is free of flavor.

According to an embodiment of the invention, the tablet is free of liquid flavor.

According to an embodiment of the invention, the tablet is free of powdered flavor.

According to an advantageous embodiment of the invention, the tablet comprises less than 0.4% by weight of high intensity sweetener, such as no more than 0.2% by weight of high intensity sweetener, such as no more than 0.1% by weight of high intensity sweetener, such as free of high intensity sweetener.

In an embodiment of the invention, the tablet comprises 0% to less than 0.4% by weight of high intensity sweetener, such as 0.01% to 0.2% by weight of high intensity sweetener, such as 0.05% to 0.1% by weight of high intensity sweetener.

According to an embodiment of the invention, the high intensity sweetener is selected from the group consisting of sucralose, aspartame, salts of acesulfame, such as acesulfame potassium, alitame, saccharin and its salts, cyclamic acid and its salts, glycyrrhizin, dihydrochalcones, thaumatin, monellin, stevioside, and any combination thereof.

According to an advantageous embodiment of the invention, the tablet is free of high intensity sweetener.

According to an advantageous embodiment of the invention, the tablet is free of flavor and free of high intensity sweetener.

In an embodiment of the invention, the sugar alcohol is a solid sugar alcohol at 25 degrees Celsius.

The solid sugar alcohols have a desirable compressibility; hence the use of solid sugar alcohols is desirable when forming compressed tablets.

It is noted that sugar alcohols, also sometimes referred to as polyols, are different from sugars.

According to an embodiment of the invention, the sugar alcohol is selected from sugar alcohols comprising at least 4 carbon atoms, such as 4 to 16 carbon atoms, such as 5 to 12 carbon atoms.

In an embodiment of the invention, the tablet is free of sugar alcohols comprising no more than three carbons. Examples of sugar alcohols comprising no more than three carbons include glycerol, propylene glycol, and ethylene glycol.

In an embodiment, the nicotine tablet is free of glycerol.

According to an advantageous embodiment of the invention, the tablet comprises the at least one sugar alcohol sugar alcohol in an amount of at least 40% by weight of the tablet, such as at least 50% by weight of the tablet, such as at least 60% by weight of the tablet, such as at least 70% by weight of the tablet, such as at least 80% by weight of the tablet.

In an embodiment of the invention, the tablet comprises the at least one sugar alcohol in an amount of 40 to 95% by weight of the tablet, such as 50 to 95% by weight of the tablet, such as 60 to 90% by weight of the tablet, such as 70 to 90% by weight of the tablet, such as 80 to 90% by weight of the tablet.

According to an advantageous embodiment of the invention, the at least one sugar alcohol comprises sugar alcohol selected from the group consisting of xylitol, maltitol, mannitol, erythritol, isomalt, sorbitol, lactitol and any combination thereof.

Thus, in the above embodiment, the at least one sugar alcohol may include mixtures of different types of sugar alcohols.

In an embodiment of the invention, the at least one sugar alcohol is selected from the group consisting of xylitol, maltitol, mannitol, erythritol, isomalt, sorbitol, lactitol and any combination thereof.

In an embodiment of the invention, the at least one sugar alcohol is selected from the group consisting of xylitol, maltitol, mannitol, isomalt, sorbitol, and any combination thereof.

In an embodiment of the invention, the at least one sugar alcohol is selected from the group consisting of xylitol, mannitol, isomalt, sorbitol and any combination thereof.

In an embodiment of the invention, the at least one sugar alcohol is selected from the group consisting of mannitol, isomalt, sorbitol and any combination thereof.

In an embodiment of the invention, the at least one sugar alcohol comprises or consists of mannitol.

In an embodiment of the invention, the at least one sugar alcohol comprises or consists of isomalt.

In an embodiment of the invention, the at least one sugar alcohol comprises or consists of sorbitol.

In an embodiment of the invention, the tablet comprises at least one sugar alcohol selected from the group consisting of xylitol, maltitol, mannitol, erythritol, isomalt, sorbitol, lactitol and any combination thereof in an amount of at least 40% by weight of the tablet, such as at least 50% by weight of the tablet, such as at least 60% by weight of the tablet, such as at least 70% by weight of the tablet, such as at least 80% by weight of the tablet.

According to the above embodiment, the tablet may comprise further sugar alcohols not selected from said group consisting of xylitol, maltitol, mannitol, erythritol, isomalt, sorbitol, lactitol and any combination thereof.

According to an advantageous embodiment of the invention, the tablet comprises said disintegrant in an amount of at least 0.5% by weight of the tablet, such as at least 1% by weight of the tablet, such as at least 2% by weight of the tablet, such as at least 3% by weight of the tablet, such as at least 5% by weight of the tablet.

According to an embodiment of the invention, the tablet comprises disintegrant in an amount of 0.5 to 15% by weight of the tablet, such as 1 to 15% by weight of the tablet, such as 2 to 14% by weight of the tablet, such as 3 to 12% by weight of the tablet, such as 5 to 10% by weight of the tablet.

According to an advantageous embodiment of the invention, said disintegrant is selected from the list consisting of starch, pregelatinated starch, modified cellulose, ion-exchange resin, calcium silicate, crosslinked cellulose, crosslinked polyvinyl pyrrolidone, crosslinked starch, crosslinked alginate, and combinations thereof.

In the present context the terms “crosslinked alginate” and “crosslinked salt of alginic acid” are used interchangeably.

According to an embodiment of the invention, the disintegrant is selected from the list consisting of starch, pregelatinated starch, cellulose, modified cellulose, microcrystalline cellulose, ion-exchange resin, calcium silicate, crosslinked cellulose, crosslinked polyvinyl pyrrolidone, crosslinked starch, crosslinked alginate, and combinations thereof.

In the present context starch refer to starches of various origin such as potato starch, corn starch, wheat starch, pea starch etc.

Examples of pregelatinized starch disintegrants include structure and brands names such as pregelatinized potato starch, pregelatinized wheat starch, pregelatinized corn starch, Lycatab®, starch 1500® etc.

Examples of modified cellulose disintegrants include structure names such as methyl cellulose, carboxymethyl cellulose, hydroxypropyl cellulose, hydroxypropyl methylcellulose etc.

According to an advantageous embodiment of the invention, said disintegrant comprises pregelatinated starch.

According to an embodiment of the invention, said disintegrant consists of pregelatinated starch.

It is understood that some disintegrants are referred to as superdisintegrants.

According to an advantageous embodiment of the invention, the disintegrant comprises a superdisintegrant.

According to an advantageous embodiment of the invention, the tablet comprises superdisintegrant in an amount of at least 0.5% by weight of the tablet, such as at least 1% by weight of the tablet, such as at least 2% by weight of the tablet, such as at least 3% by weight of the tablet, such as at least 5% by weight of the tablet.

According to an embodiment of the invention, the tablet comprises superdisintegrant in an amount of 0.5 to 15% by weight of the tablet, such as 1 to 15% by weight of the tablet, such as 2 to 14% by weight of the tablet, such as 3 to 12% by weight of the tablet, such as 5 to 10% by weight of the tablet.

In some embodiments, the disintegrant may comprise combination of regular disintegrant and superdisintegrant.

In an embodiment of the invention, the disintegrant consists of superdisintegrant.

According to an advantageous embodiment of the invention, the superdisintegrant is selected from the group consisting of crosslinked cellulose, crosslinked polyvinyl pyrrolidone, crosslinked starch, crosslinked alginate, and any combinations thereof.

Examples of crosslinked cellulose super disintegrants include structure and brands names such as cross-linked sodium carboxymethylcellulose, Croscarmellose®, Ac-Di-Sol®, Solutab® etc.

Examples of crosslinked polyvinyl pyrrolidone (PVP) super disintegrants include structure and brands names such as Kollidon®, Polyplasdone®, polyplasdone XL®, Kollidon CL® etc. Crosslinked polyvinyl pyrrolidone (PVP) disintegrant is also sometimes referred to as crospovidone.

Examples of crosslinked starch super disintegrants include structure and brands names such as sodium starch glycolate, Glycolys®, Explotab®, Primogel®, Vivastar®, Tablo® etc.

According to an advantageous embodiment of the invention, the disintegrant is a super disintegrant selected from the group consisting of crosslinked cellulose, crosslinked polyvinyl pyrrolidone (PVP), crosslinked starch, and any combinations thereof.

According to an advantageous embodiment of the invention, the disintegrant is a super disintegrant selected from the group consisting of crosslinked polyvinyl pyrrolidone, croscarmellose, sodium starch glycolate, and any combinations thereof.

In an embodiment of the invention, the disintegrant comprises cross-linked polyvinylpyrrolidone.

In an embodiment of the invention, at least 50% by weight of the cross-linked polyvinylpyrrolidone has a particle size below 50 micrometers, such as 50 to 100% by weight of the cross-linked polyvinylpyrrolidone has a particle size below 50 micrometers.

In an embodiment of the invention, at least 25% by weight of the cross-linked polyvinylpyrrolidone has a particle size below 15 micrometers, such as 25 to 100% by weight of the cross-linked polyvinylpyrrolidone has a particle size below 15 micrometers.

In an embodiment of the invention, ready to use systems may be used. Typically, such ready-to-use systems may e.g. replace filler, disintegrant, glidant or similar with a single powder mix. Suitable ready-to-use systems for the purpose, but not limited to, include Pearlitol Flash (Roquette), Pharmaburst 500 (SPI Pharma), Ludiflash (BASF), ProSolv (JRS Pharma), ProSolv EasyTab (JRS Pharma), F-Melt (Fuji Chemical), SmartEx50 or SmartEx100 (Shin Etsu/Harke Pharma).

Thus, in the above embodiment, ready to use systems may be included as a convenient way of including e.g. both sugar alcohol and disintegrant.

According to an advantageous embodiment of the invention, the tablet is a compressed tablet.

Thus, in the above embodiment, the tablet is a compressed orally disintegrating nicotine tablet, also referred to as a compressed nicotine ODT.

According to an advantageous embodiment of the invention, the tablet is composed of a plurality of compressed particles.

According to an advantageous embodiment of the invention, the tablet is compressed using a compression force of 1 to 35 kN, such as 2 to 30 kN, such as 2-20 kN

According to an advantageous embodiment of the invention, the nicotine is selected from the group consisting of a nicotine salt, nicotine free base, a nicotine-ion exchange resin combination, a nicotine inclusion complex or nicotine in any non-covalent binding, nicotine bound to zeolites, nicotine bound to cellulose, nicotine bound to starch microspheres, and any combination thereof.

In an embodiment of the invention, the nicotine is selected from the list consisting of nicotine free base and nicotine salts, or combinations thereof. It is noted that when nicotine free base and/or nicotine salt is used, it is implied that the nicotine is provided without a carrier unless explicitly specified otherwise.

According to an advantageous embodiment of the invention, the nicotine comprises nicotine free base.

Free base nicotine includes nicotine mixed with sugar alcohols, modified calcium carbonate, water-soluble fibers, water-insoluble fibers, and combinations thereof.

In an embodiment of the invention, the nicotine is nicotine free base.

In an embodiment of the invention, the nicotine comprises a nicotine inclusion complex. In an embodiment of the invention, the nicotine comprises nicotine bound to an ion exchange resin. In an embodiment of the invention, the nicotine comprises nicotine bound to polacrilex resin. As used herein, the term “NPR” refers to nicotine bound to polacrilex resin.

According to an advantageous embodiment of the invention, the nicotine comprises nicotine salt.

An advantage of the above embodiment may be that a fast craving relief of nicotine may be facilitated, e.g. due to a fast dissociation of the nicotine salt.

In an embodiment of the invention, the nicotine is a nicotine salt.

According to an advantageous embodiment of the invention, the nicotine salt is selected from the group consisting of nicotine ascorbate, nicotine aspartate, nicotine benzoate, nicotine monotartrate, nicotine bitartrate, nicotine chloride, nicotine citrate, nicotine fumarate, nicotine lactate, nicotine mucate, nicotine laurate, nicotine levulinate, nicotine malate, nicotine perchlorate, nicotine pyruvate, nicotine salicylate, nicotine sorbate, nicotine succinate, nicotine sulfate, and any combination thereof.

It is understood that the nicotine salt may also be provided as a hydrated salt, or as a combination of hydrated and non-hydrated salt.

Nicotine chloride may e.g. be nicotine hydrochloride and/or nicotine dihydrochloride.

In an embodiment of the invention, the nicotine salt is selected from the list consisting of nicotine benzoate, nicotine monotartrate, nicotine bitartrate, nicotine hydrochloride, nicotine dihydrochloride, nicotine lactate, nicotine malate, nicotine pyruvate, nicotine succinate, and combinations thereof.

According to an advantageous embodiment of the invention, the nicotine comprises nicotine bitartrate.

The term NBT may be used as an abbreviation to denote nicotine bitartrate.

An advantage of the above embodiment may be that a fast craving relief of nicotine may be facilitated, e.g. due to a fast dissociation of nicotine bitartrate.

In an embodiment of the invention, the nicotine is nicotine bitartrate.

In an embodiment of the invention, said nicotine is provided in ionic complex with at least one mucoadhesive water-soluble anionic polymer.

In an embodiment of the invention, the nicotine comprises synthetic nicotine.

In an embodiment of the invention, the nicotine consists of synthetic nicotine.

According to an advantageous embodiment of the invention, the tablet comprises nicotine in an amount of at least 0.2 mg, such as at least 0.5 mg, such as at least 1.0 mg.

In an embodiment of the invention, the tablet comprises nicotine in an amount of 0.2 mg to 12.0 mg, such as 0.2 mg to 10.0 mg, such as 0.5 mg to 8.0 mg, such as 0.5 mg to 6.0 mg, such as 1.0 mg to 4.0 mg, such as 1.0 to 3.0 mg, such as 1.0 to 2.0 mg.

According to an advantageous embodiment of the invention, the tablet comprises nicotine in an amount of at least 0.2% by weight of the tablet, such as at least 0.3% by weight of the tablet, such as at least 0.5% by weight of the tablet.

In an embodiment of the invention, the tablet comprises nicotine in an amount of 0.2 to 5% by weight of the tablet, such as 0.3 to 3% by weight of the tablet, such as 0.5 to 2% by weight of the tablet.

According to an advantageous embodiment of the invention, the tablet comprises a pH regulating agent.

According to an advantageous embodiment of the invention, the pH regulating agent is an alkaline pH regulating agent, such as an alkaline buffering agent.

In an embodiment of the invention, the pH regulating agent is free of acidic pH regulating agent.

In an embodiment of the invention, the pH regulating agent is a buffering agent.

According to an advantageous embodiment of the invention, the pH regulating agent comprises pH regulating agent selected from the group consisting of sodium carbonate, sodium bicarbonate, potassium carbonate, potassium bicarbonate, trometamol, amino acids, di-alkali hydrogen phosphate, tri-alkali phosphate, or any combination thereof.

In an embodiment of the invention, the pH regulating agent is selected from the group consisting of sodium carbonate, sodium bicarbonate, potassium carbonate, potassium bicarbonate, trometamol, amino acids, disodium hydrogen phosphate, dipotassium hydrogen phosphate, trisodium phosphate, tripotassium phosphate, or any combination thereof.

In an embodiment of the invention, the pH regulating agent is selected from the group consisting of sodium carbonate, sodium bicarbonate, potassium carbonate, potassium bicarbonate, trometamol, amino acids, or any combination thereof.

According to an advantageous embodiment of the invention, the pH regulating agent is selected from the group consisting of sodium carbonate, sodium bicarbonate, potassium carbonate, potassium bicarbonate, or any combination thereof.

Combinations of a carbonate and a bicarbonate may be especially advantageous. Such combination may e.g. be a sodium carbonate-sodium bicarbonate buffer system, e.g. sodium carbonate and sodium bicarbonate in a weight-ratio between 5:1 and 1:1, preferably in a weight-ratio between 4.1:1 and 1.5:1.

According to an advantageous embodiment of the invention, the pH regulating agent comprises or consists of sodium carbonate.

An advantage of the above embodiment may be an increased uptake of nicotine in the oral cavity.

In an embodiment of the invention, the pH regulating agent is selected from the group consisting of trometamol, amino acids, disodium hydrogen phosphate, dipotassium hydrogen phosphate, trisodium phosphate, tripotassium phosphate, or any combination thereof.

In an embodiment of the invention, the pH regulating agent is selected from the group consisting of trometamol, disodium hydrogen phosphate, dipotassium hydrogen phosphate, trisodium phosphate, tripotassium phosphate, or any combination thereof.

In an embodiment of the invention, the pH regulating agent comprises di-alkali hydrogen phosphate and/or tri-alkali phosphate, such as disodium phosphate, dipotassium phosphate, trisodium phosphate and/or tripotassium phosphate.

Trometamol and phosphate buffers have a desirable relative neutral taste, hence the use of these pH regulating agents may be found not compromise the taste and mouthfeel of the nicotine tablet.

In an embodiment of the invention, the pH regulating agent comprises trometamol.

In an embodiment of the invention, the pH regulating agent consists of trometamol.

In the present context the term trometamol refers to (tris(hydroxymethyl)aminomethane), also sometimes referred to as tris buffer.

In an embodiment of the invention, the pH regulating agent comprises amino acid.

In an embodiment of the invention, the pH regulating agent consists of amino acid.

According to an advantageous embodiment of the invention, the tablet comprises the pH regulating agent in an amount of at least 0.2% by weight of the tablet, such as at least 0.5% by weight of the tablet, such as at least 1% by weight of the tablet, such as at least 2% by weight of the tablet.

In an embodiment of the invention, the tablet comprises the pH regulating agent in an amount of 0.2 to 10% by weight of the tablet, such as 0.5 to 8% by weight of the tablet, such as 1 to 6% by weight of the tablet, such as 2 to 4% by weight of the tablet.

According to an advantageous embodiment of the invention, the tablet comprises the pH regulating agent in an amount of at most 2% by weight of the tablet, such as at most 1.5% by weight of the tablet, such as at most 1% by weight of the tablet, such as at most 0.5% by weight of the tablet, such as at most 0.2% by weight of the tablet, such as at most 0.1% % by weight of the tablet.

According to an advantageous embodiment of the invention, the tablet is free of effervescent agents.

An advantage of the above embodiment may be an improved user experience. Often, inclusion of effervescent agents may lead to tingling sensations, which may be rather undesirable, in particular if the tablet is administered between the gum and the lip.

A further advantage may be that using effervescent agents may lead to the oral pH value being below the optimum range for efficient nicotine absorption over the mucous membrane. Avoiding the use of effervescent agents may therefore facilitate improved nicotine absorption.

According to an advantageous embodiment of the invention, the tablet has a weight of no more than 600 mg, such as no more than 500 mg, such as no more than 400 mg, such as no more than 300 mg, such as no more than 200 mg, such as no more than 150 mg, such as no more than 120 mg, such as no more than 100 mg, such as no more than 50 mg.

In an embodiment of the invention, the tablet has a weight of 25 to 600 mg, such as 25 to 500 mg, such as 25 to 400 mg, such as 25 to 300 mg, such as 25 to 200 mg, such as 50 to 150 mg, such as 70-120 mg, such as about 75 mg or about 100 mg, or such as 25 to 50 mg.

According to an advantageous embodiment of the invention, the tablet is a multilayer tablet comprising a first layer and a second layer.

According to an embodiment of the invention, the first and the second layers are fused by compression.

According to an advantageous embodiment of the invention, the tablet is a one-layer tablet.

In the present context the term layer refers to a module of the tablet composing a significant part of the tablet, such as e.g. at least 20% by weight of the tablet, such as at least 30% by weight of the tablet, such as at least 40% by weight of the tablet.

In an embodiment of the invention, the tablet is a multilayer tablet. The multilayer tablet may comprise three or more layers.

According to an advantageous embodiment of the invention, the tablet further comprises dissolution modifiers in an amount of at most 2% by weight of the tablet, such as at most 1% by weight of the tablet, such as at most 0.5% by weight of the tablet, such at most 0.25% by weight of the tablet, such at most 0.1% by weight of the tablet.

According to an advantageous embodiment of the invention, the dissolution modifiers are selected xanthan gum, guar gum, konjac gum, tara gum, gellan gum, locust bean gum, gum arabic, monovalent alginate, pullulan, tragacanth gum, gum karaya, fenugreek gum, cassia gum, carrageenan, agar, alginic acid and monovalent salts thereof, and any combination thereof.

It is noted that in the present context the terms “monovalent alginate”, “monovalent salt of alginate” and “monovalent salt of alginic acid” are used interchangeably. It is further noted that in the present context dissolution modifiers comprise non-crosslinked alginates, whereas dissolution modifiers do not comprise crosslinked alginates.

An advantage of the above embodiment may be that the small amount of dissolution modifiers may improve the mouthfeel.

According to an advantageous embodiment of the invention, the dissolution modifiers are selected from the group consisting of xanthan gum, guar gum, and any combinations thereof.

An advantage of the above embodiment may be that the small amount of dissolution modifiers may improve the mouthfeel.

In an embodiment of the invention, the dissolution modifier comprises xanthan gum.

In an embodiment of the invention, the dissolution modifier comprises guar gum.

According to an advantageous embodiment of the invention, the tablet is free of dissolution modifiers.

In an embodiment of the invention, the tablet is substantially free of dissolution modifiers, such as free of dissolution modifiers.

In an embodiment of the invention, the tablet is free of xanthan gum.

According to an advantageous embodiment of the invention, the tablet is free of mucoadhesives.

Thus, according to an embodiment of the invention, the tablet is not an adhering tablet, e.g. by not including any mucoadhesive, or by only including a minor amount of mucoadhesive, such as no more than 10% by weight of the tablet, such as no more than 5% by weight of the tablet, such as no more than 2% by weight of the tablet, such as no more than 1% by weight of the tablet.

According to an advantageous embodiment of the invention, the tablet comprises a lubricant, such as sodium stearyl fumarate (SSF) or magnesium stearate.

According to an advantageous embodiment of the invention, the tablet is a non-chewable tablet.

It is noted that that the tablet of the above embodiment is non-chewable in the sense that it is not intended for chewing.

The invention further relates to an orally disintegrating nicotine tablet for fast nicotine craving relief, the tablet comprising nicotine, disintegrant, and at least one sugar alcohol, wherein the tablet has a total amount of flavor and high intensity sweetener of less than 0.8% by weight of the tablet.

According to an advantageous embodiment of the invention, the tablet has a total amount of flavor and high intensity sweetener of no more than 0.6% by weight of the tablet, such as no more than 0.4% by weight of the tablet, such as no more than 0.3% by weight of the tablet, such as no more than 0.2% by weight of the tablet.

In an embodiment of the invention, the tablet has a total amount of flavor and high intensity sweetener of 0 to 0.8% by weight of the tablet, such as 0.01 to 0.6% by weight of the tablet, such as 0.05 to 0.4% by weight of the tablet, such as 0.1 to 0.3% by weight of the tablet, such as 0.1 to 0.2% by weight of the tablet.

The invention further relates to an oral nicotine tablet comprising the orally disintegrating nicotine tablet according to any of claims 1-Y as a first module and further comprising a second module, wherein the second module is different in composition compared to the first module.

Thus, in the present context it is understood that the second module has a composition that is different from the composition of the first module.

According to an advantageous embodiment of the invention, the second module is a lozenge module.

According to an advantageous embodiment of the invention, the second module comprises at least one sugar alcohol.

According to an advantageous embodiment of the invention, the second module comprises a dissolution modifier.

According to an advantageous embodiment of the invention, the second module comprises the at least one sugar alcohol in an amount of at least 40% by weight of the second module, such as at least 50% by weight of the second module, such as at least 60% by weight of the second module, such as at least 70% by weight of the second module, such as at least 80% by weight of the second module.

In an embodiment of the invention, the second module comprises the at least one sugar alcohol in an amount of 40 to 95% by weight of the second module, such as 50 to 95% by weight of the second module, such as 60 to 90% by weight of the second module, such as 70 to 90% by weight of the second module, such as 80 to 90% by weight of the second module.

According to an advantageous embodiment of the invention, the at least one sugar alcohol of the second module comprises sugar alcohol selected from the group consisting of xylitol, maltitol, mannitol, erythritol, isomalt, sorbitol, lactitol and any combination thereof.

According to an advantageous embodiment of the invention, the second module comprises dissolution modifier in an amount of at least 1% by weight of the second module, such as at least 2% by weight of the second module, such as at least 3% by weight of the second module.

In an embodiment of the invention, the second module comprises dissolution modifier in an amount of 1 to 15% by weight of the second module, such as 2 to 10% by weight of the second module, such as 3 to 8% by weight of the second module.

According to an embodiment of the invention, the dissolution modifiers are selected from the group consisting of xanthan gum, guar gum, konjac gum, tara gum, gellan gum, locust bean gum, gum arabic, alginate, pullulan, tragacanth gum, gum karaya, fenugreek gum, cassia gum, carrageenan, agar, alginic acid and monovalent salts thereof, and any combination thereof.

According to an embodiment of the invention, the dissolution modifiers are selected from the group consisting of xanthan gum, guar gum, and any combinations thereof.

According to an advantageous embodiment of the invention, the second module comprises nicotine.

According to an advantageous embodiment of the invention, the second module comprises less than 0.4% by weight of flavor.

In an embodiment of the invention, the second module comprises 0 to 0.4% by weight of flavor, such as 0.05 to 0.4% by weight of flavor.

According to an advantageous embodiment of the invention, the second module is free of flavor.

According to an advantageous embodiment of the invention, the tablet is compressed.

Thus, in the above embodiment, both the first and the second module is compressed, i.e. the tablet is composed of a plurality of compressed particles.

In an embodiment of the invention, the first and second module are layers. Thus, in this embodiment, the first module is a first layer, and the second module is a second layer.

### DETAILED DESCRIPTION

As used herein, the term “orally disintegrating tablet” refers to a tablet for oral administering which disintegrates in the oral cavity relatively fast from the administering, such as within 60 seconds.

Orally disintegrating tablets may also be referred to “orally dissolving tablets”, and these two terms are used interchangeably herein. Commonly, these terms are also referred to by their abbreviation, ODT. Similarly, the terms “fast dissolving tablet” and “fast disintegrating tablet”, as well as the abbreviation FDT, refers herein to an orally disintegrating tablet. It is noted that disintegrating tablets are considered fully disintegrable, i.e. that they completely disintegrate when administered to the oral cavity. In some embodiments, the disintegrating tablets are composed solely of water-soluble substances and are therefore completely water-soluble. In other embodiments, the disintegrating tablets comprise some amounts of water-insoluble compositions, as long as these do not prevent the tablet from fully disintegrating. Thus, it should be understood that a disintegrating tablet does not include e.g. chewing gums, which do not completely disintegrate or dissolve, and further that it does not include hard boiled candy, i.e. tablets based on cooked sugar and/or sugar alcohol and which only slowly dissolves instead of disintegrating.

As used herein, the term “disintegrate” refers to a reduction of a said object to components, fragments or particles. Disintegration may both refer to in vivo disintegration, the methods of which are defined herein, or in vitro disintegration. The in vitro measurements are carried out in accordance with European Pharmacopeia 9.0, section 2.9.1, Disintegration of tablets and capsules.

As used herein, the term “dissolve” is the process where a solid substance enters a solvent (oral saliva) to yield a solution. Unless otherwise stated, dissolving implies a full dissolving of the compound in question.

As used herein, the terms “disintegrant” refers to an ingredient facilitating disintegration of a disintegrating tablet, when the disintegrating tablet comes into contact with saliva. Disintegrants usable within the scope of the invention may include starch, pregelatinated starch, modified starch (including potato starch, maize starch, starch 1500, sodium starch glycolate and starch derivatives), crosslinked alginates, ion-exchange resin, and superdisintegrants, such as crosslinked cellulose (such as sodium carboxy methyl cellulose), crosslinked polyvinyl pyrrolidone (PVP), crosslinked starch, crosslinked alginic acid, natural superdisintegrants, and calcium silicate. Disintegrants may often be considered as measure promoting the break-up of the dosage form into smaller fragments upon administration to allow the onset of drug dissolution and eventual absorption.

As used herein, the term “nicotine” refers to nicotine in any form, including free base nicotine, nicotine salts, nicotine bound to ion exchange resins, such as nicotine polacrilex, nicotine bound to zeolites; nicotine bound to cellulose, such as microcrystalline cellulose, such as of microbial origin, or starch microspheres, nicotine bound to CaCO3, and mixtures thereof. Thus, when referring to nicotine amounts, the amounts refers to the amount of pure nicotine. Thus, when measuring the concentration of nicotine added as nicotine salt, it is the mass of the equivalent amount of pure nicotine, not the mass of the salt, that is relevant. Nicotine also covers nicotine not obtained from tobacco, often referred to as synthetic nicotine.

As used herein, the term “nicotine salt” refers to nicotine in ionized form bonded electrostatically to a counterion.

As used herein, the term “NBT” refers to nicotine bitartrate and hydrates thereof.

As used herein, the term “%” and “percent” refers to percent by weight, unless otherwise is stated.

As used herein, the term “pH regulating agent” refers to agents, which active adjust and regulates the pH value of the solution to which they have been added or are to be added. Thus, pH regulating agents may be acids and bases, including acidic buffering agents and alkaline buffering agents. On the other hand, pH regulating agents does not include substances and compositions that can only affect the pH by dilution. Furthermore, pH regulating agents does not include e.g, flavoring, fillers, etc. In an advantageous embodiment of the invention, the pH regulating agent is alkaline.

As used herein, the term “buffering agent” is used interchangeably with “buffer” and refers to agents for obtaining a buffer solution. Buffering agents may include alkaline buffering agents, i.e. for obtaining a buffer solution with an alkaline pH.

In an embodiment of the invention, the tablet comprises a filler. In an embodiment of the invention, the filler is selected form the group consisting of microcrystalline cellulose, magnesium- and calcium carbonate, sodium sulphate, ground limestone, silicate compounds such as magnesium- and aluminum silicate, kaolin and clay, aluminum oxide, silicon oxide, talc, titanium oxide, mono-, di- and tri-calcium phos-phates, cellulose polymers, such as wood, starch polymers, fibers and combinations thereof.

In an embodiment of the invention, the tablet comprises a bulk sweetener. In an embodiment of the invention, the bulk sweetener comprises or consists of sugar sweetener and/or sugarless sweetener.

The bulk sweeteners may often support the flavor profile of the formulation.

In an embodiment of the invention, the sugar sweetener is selected from saccharide-containing components, such as sucrose, dextrose, maltose, saccharose, lactose, sorbose, dextrin, trehalose, D-tagatose, dried invert sugar, fructose, levulose, galactose, and the like, alone or in combination. These sugar sweeteners may also be included as a humectant.

In an embodiment of the invention, the sugarless sweeteners is selected from sugar alcohols (also sometimes referred to as polyols), such as sorbitol, erythritol, xylitol, maltitol, mannitol, lactitol, and isomalt.

In an embodiment of the invention, the disintegrant is selected from crospovidone, croscarmellose sodium, and sodium starch glycolate. In an embodiment of the invention, the disintegrant is selected from starch, pregelatinated starch, modified starch (including potato starch, maize starch, starch 1500, sodium starch glycolate and starch derivatives), ion-exchange resin, and superdisintegrants, such as crosslinked cellulose (such as sodium carboxy methyl cellulose), crosslinked polyvinyl pyrrolidone (PVP), crosslinked starch, crosslinked alginate, natural superdisintegrants, and calcium silicate, and combinations thereof.

In an embodiment of the invention, the tablet comprises a high intensity sweetener. In an embodiment of the invention, the high intensity sweetener is selected from sucralose, aspartame, salts of acesulfame, such as acesulfame potassium, alitame, saccharin and its salts, cyclamic acid and its salts, glycyrrhizin, dihydrochalcones, thaumatin, monellin, stevioside and the like, alone or in combination. In an embodiment of the invention, the tablet is free of high intensity sweetener.

In an embodiment of the invention, the tablet comprises one or more flavors. In an embodiment of the invention, one or more flavors are selected from peppermint, menthol, almond, almond amaretto, apple, Bavarian cream, black cherry, black sesame seed, blueberry, brown sugar, bubblegum, butterscotch, cappuccino, caramel, caramel cappuccino, cheesecake (graham crust), cinnamon redhots, cotton candy, circus cotton candy, clove, coconut, coffee, clear coffee, double chocolate, energy cow, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry kiwi, tropical punch, tutti frutti, vanilla, or any combination thereof.

According to an embodiment of the invention, flavor may be used as taste masking for the nicotine.

In an embodiment of the invention, the pH regulating agent is a buffering agent. In an embodiment of the invention, the buffering agent is sodium carbonate. In an embodiment of the invention, the buffering agents is selected from carbonate, including monocarbonate, bicarbonate and sesquicarbonate, glycerinate, phosphate, glycerophosphate, acetate, glyconate or citrate of an alkali metal, ammonium, tris buffer, amino acids and mixtures thereof. Encapsulated buffer such as Effersoda may also be used.

Buffering agent in the tablet may be used to obtain the desired pH-values in the saliva of a tablet user.

In some embodiments, the buffering agent comprises sodium carbonate and sodium bicarbonate, e.g. in a weight-ratio between 5:1 and 2.5:1, preferably in a weight-ratio between 4.1:1 and 3.5:1.

A high suitable buffering agent according to advantageous embodiments of the present invention is the sodium carbonate-sodium bicarbonate buffer system.

In an embodiment of the invention, silicon dioxide is used as a glidant. Other glidants usable for the formulation may also be used within the scope of the invention.

In an embodiment of the invention, magnesium stearate is used as a lubricant. Other lubricants usable for the formulation may also be used within the scope of the invention.

In an embodiment of the invention, ready to use systems may be used. Typically, such ready-to-use systems may e.g. replace filler, disintegrant, glidant or similar with a single powder mix. Suitable ready-to-use systems for the purpose, but not limited to, include Pearlitol Flash (Roquette), Pharmaburst 500 (SPI Pharma), Ludiflash (BASF), ProSolv (JRS Pharma), ProSolv EasyTab (JRS Pharma), F-Melt (Fuji Chemical), SmartEx50 or SmartEx100 (Shin Etsu/Harke Pharma).

In order to obtain an orally disintegrating tablet, such as an orally disintegrating tablet disintegrating within 60 seconds from oral administration on the tongue, or a disintegrating tablet disintegrating within 60 seconds in vitro, a range of parameters can be adjusted.

First, by varying the composition, the disintegration time can be altered. Using ingredients with a high water-solubility may facilitate a lowered disintegration time.

Particularly, by varying the amount and type of the disintegrant disintegrant may significantly influence the disintegration time, the disintegration time may be further adjusted. For example, if a tablet having a lower disintegration time is desired, the percentage content of disintegrant may be increased and/or the type of disintegrant may be at least partly exchanged for a more effective disintegrant. In this respect, superdisintegrants are generally considered more effective disintegrants than disintegrants not being superdisintegrants.

Additionally, including a dissolution modifier may significantly influence the disintegration time, by varying the amount and type of the dissolution modifier. For example, if a shorter disintegration time is desired, the content of dissolution modifier may be decreased (or even eliminated) and/or the type of dissolution modifier may be at least partly exchanged for a dissolution modifier less effective in delaying dissolution.

Also, decreasing the particle size of the disintegrant tends to lower the disintegration time, likely due to an increased surface area to volume ratio.

Furthermore, the compression force used in compressed tablets correlate significantly with the obtained hardness, such that a high compression force typically increases the hardness of the obtained tablet. By adjusting the hardness of a tablet, the disintegration time may also be influenced, such that a lowered hardness typically gives a shorter disintegration time. Here it has been observed for a number of compositions that by applying the correct compression force a disintegration time below 60 seconds upon oral administration and/or 60 seconds in vitro can be achieved, whereas a too high compression force may result in a longer disintegration time above 60 seconds in vivo or above 60 seconds in vitro. In this regard it is noted that the threshold compression force may vary significantly, depending on other parameters, such as overall composition, content and type of disintegrant, etc. When, for example, a certain setup results in a too slow disintegration, a further way of adjusting may be to replace a regular disintegrant with a superdisintegrant, i.e. which facilitates disintegration in a more efficient way.

Increasing the water-solubility may also be facilitated by exchanging ingredients with low water-solubility with ingredients having higher water-solubility. For example, using sugar alcohols as fillers may be very advantageous insofar that the sugar alcohols have a higher water solubility than alternative fillers.

Moreover, using sugar alcohols with a lower compactibility leads to lower disintegration time. Too low compactibility may compromise the mechanical strength of the tablet and lead to undesirably high friability and risk of cracks etc.

Further examples of parameters that may be adjusted in order to obtain an orally disintegrating nicotine tablet include size and shape of the tablet. The larger the tablet, the longer the disintegration time and thus release of the contents of the tablet, such as release of nicotine.

Further, increasing the flatness (e.g. quantified by a diameter to height ratio) for a disc-shaped tablet typically increases disintegration time by increasing the surface-to-volume. As long as the tablet has a satisfactory mechanical strength, flatness may be increased.

Also, modifying the cross-sectional profile from a convex type tablet to a concave shaped tablet lowers the disintegration time. It is noted that this may to some degree lower the mechanical strength of the tablet, however, as long as it is satisfactory, pursuing the concave cross-section may help to increase disintegration and thus lower the disintegration time.

Also, when using binders, e.g. to obtain a higher cohesiveness and mechanical strength of the tablet, the amount of such binders may be decreased as much as possible to obtain a higher disintegration rate and thus a shorter disintegration time.

Furthermore, by adding a salivation agent to the tablet, an increased amount of saliva in the vicinity of the tablet may be facilitated, which again supports the dissolving and disintegration of the tablet to reduce the disintegration time. Thus, sweeteners, in particular sugar alcohols, may be advantageous in this respect.

Further, the type and amount of lubricant, if any, may be adjusted to optimize disintegration time. For example, using Sodium stearyl fumarate (SSF) typically leads to a lower disintegration time compared to when using magnesium stearate MgSt.

Thus, a wide range of parameters may be adjusted when designing an orally disintegrating nicotine tablet designed for disintegrating within the desired time period, such as within a period of 60 second in vitro or within 60 seconds from administration on the tongue.

Typically, the formulation comprises of ingredients selected from the group consisting of bulk sweeteners, fillers, ready to use systems, flavors, dry-binders, disintegrant, hereunder superdisintegrants, tabletting aids, anti-caking agents, emulsifiers, antioxidants, enhancers, absorption enhancers, buffering agents, high intensity sweeteners, colors, glidants, lubricants, or any combination thereof. Absorption enhancers may include e.g. pH regulating agents, such as buffering agents, and mucoadhesive.

In an embodiment of the invention, the tablet core is provided with an outer coating.

In an embodiment of the invention, said outer coating is selected from the group consisting of hard coating, soft coating and edible film-coating or any combination thereof.

According to an embodiment of the invention, at least a part of the nicotine is adhered to dry-binder particles.

According to an embodiment of the invention, an amount of dry-binder is used to adhere nicotine to bulk sweetener.

According to an embodiment of the invention, said disintegrating tablet comprises one or more encapsulation delivery systems.

According to an embodiment of the invention, the nicotine tablet may act as a first module, wherein the tablet further comprises a module with a different composition. The further module may be a slowly dissolvable lozenge module. The tablet of course does not comprise e.g. chewing gum modules that does not dissolve in water. Furthermore, the lozenge is water-dissolvable in the sense that it disintegrates and that main constituents dissolve in water. The nicotine tablet according to the above embodiment may in an embodiment be a compressed tablet, formed by compression of at least a first powdered composition and a second powdered composition to give the first and second module, respectively. The tablet may comprise some amounts of water-insoluble material, such as e.g. MCC. For example, the tablet may dissolve within a period of at least 2 minutes upon oral administration, such as at least 3 minutes, such as at least 4 minutes, such as at least 5 minutes.

As used herein the term “lozenge module” refers to a module imparting lozenge properties, i.e. a module that dissolve or disintegrate slowly in the mouth, whereby its constituents are slowly released, e.g. pH regulating agents, flavor, nicotine etc. depending on the specific embodiment. For example, the lozenge module may dissolve within a period of at least 2 minutes upon oral administration, such as at least 3 minutes, such as at least 4 minutes, such as at least 5 minutes.

A module in the present context thus, in an advantageous embodiment, encompasses at least 10% by weight of the tablet. For compressed oral nicotine tablets, a module may thus encompass a population of compressed particles weighing at least 10% by weight of the tablet. In other words, a module is not intended to refer to individual particles as conventionally understood from the art of tableting.

### EXAMPLES

### Example 1

### Preparation of One Layer Tablets

Raw materials are weighed from bags or buckets into separate weighing containers.

All excipients are sifted through an 800 micrometer sieve into a stainless steel or plastic bin in the following order:
Half the filler/bulk sweetener
The API and all other excipients, except lubricant (if any)
The remaining half of the filler/bulk sweetener

These are mixed in a Turbula mixer for 4-10 minutes at 25 RPM. Then lubricant, for example magnesium stearate is sifted through an 800 micrometer sieve into the mixing bin, and the lubrication is conducted by additional mixing for 1-2 minutes at 25 RPM. The fill level of the mixing bin is kept between 40% and 70%, according to standardized practice. The lubricated powder blend is transferred to the hopper of a tableting machine.

The orally disintegrating tablets are manufactured on a lab scale machine, for example RIVA Piccola bi-layer tablet press. The tablet machine is commissioned by adjusting the fill depth and compression force so the weight and hardness of the tablets match the acceptance criteria. A pre-compression force could be included to avoid capping.

TABLE 1
Suggested start up parameters.

| Parameter | Target value | |
|---|---|---|
| Speed | 10-20 | rpm |
| Weight of ODT | 100 mg +/− 5% | |
| Compression force | 2-8 | kN |

| | |
|---|---|
| Thickness | N/A* |
| Friability (100 rpm) | <1% |

*The design of punches is not fixed. As the curvature impacts thickness, the thickness is not a fixed target at this time of development.

The acceptance criteria for friability should be fulfilled so packaging of the resulting orally disintegrating tablets is possible, but in this embodiment, the bulk sweetener and or filler should have relatively good compressibility and still have fast disintegration. The orally disintegrating tablets according to the invention may comprise coloring agents. According to an embodiment of the invention, the orally disintegrating tablets may comprise color agents and whiteners such as FD&C-type dyes and lakes, fruit and vegetable extracts, titanium dioxide and combinations thereof.

### Example 2

### Preparation of Tablets Comprising a First and a Second Modules.

The composition of second module, i.e. the module that is different in composition from the orally disintegrating tablet module, is prepared by pouring about half the sugar alcohol into a mixing bowl, followed by the other ingredients except lubricant, and finally the remaining sugar alcohol. The ingredients are tumbled/mixed with a mixer (Turbula or Duma) for 4-10 min at 49 rpm.

Lubricant is added and the ingredients are further mixed for 1-2 min at 49 rpm.

The composition of first module, i.e. the orally disintegrating tablet module, is prepared by pouring all the ingredients except lubricant, into a mixing bowl. The ingredients are tumbled/mixed with a mixer (Turbula or Duma) for 4-10 min at 49 rpm.

Lubricant is added and the ingredients are further mixed for 1-2 min at 49 rpm.

The lubricated powder blends are sequentially transferred to the hopper of a tableting machine.

The second module is then compressed at a compression force of about 3-8 kN, after which the first module is fused by compression to the second module at a compression force of about 20-30 kN. Punch used unless otherwise specified: 12.00 mm, circular, shallow concave, B tooling.

The tablets are manufactured on a lab scale machine, for example RIVA Piccola tablet press. The tablet machine is commissioned by adjusting the fill depth and compression force so the weight and hardness of tablets match the acceptance criteria. A pre-compression force could be included to avoid capping.

### Example 3: Tablet Compositions, One Layer Tablets

### Example 3A

Tablets were prepared according to example 1, unless otherwise specified.

In this example, the following conditions were applied. Punch used: 7.00 mm, circular, shallow concave, B tooling. Tablet weight: 100.0 mg.

TABLE 2
Composition of tablets.

| | LFT(1) | LFT(2) | LFT(3) | LFT(4) |
|---|---|---|---|---|
| Nicotine dose [mg] | 2.0 | 2.0 | 2.0 | 2.0 |
| Raw material | Content in weight percent | | | |
| Sorbitol | 76.76 | — | — | — |
| Isomalt | — | 76.76 | — | — |
| Xylitol | — | — | 76.76 | — |
| Mannitol | — | — | — | 76.76 |
| NBT | 6.14 | 6.14 | 6.14 | 6.14 |
| Microcrystalline cellulose | 2.1 | 2.1 | 2.1 | 2.1 |
| Guar gum | 0.4 | 0.4 | 0.4 | 0.4 |
| Sodium carbonate | 5 | 5 | 5 | 5 |
| Crospovidone | 8 | 8 | 8 | 8 |
| Flavor | 0.3 | 0.3 | 0.3 | 0.3 |
| HIS | 0.3 | 0.3 | 0.3 | 0.3 |
| Sodium stearyl fumerate | 1 | 1 | 1 | 1 |
| Total | 100 | 100 | 100 | 100 |

NBT = nicotine bi-tartrate (nicotine content of 32.38% by weight). MCC = microcrystalline cellulose. HIS = High intensity sweeteners.

Preferred high intensity sweeteners (HIS) may e.g. be sucralose, acesulfame potassium, and mixtures thereof. Other high intensity sweeteners, such as aspartame, salts of acesulfame, such as acesulfame potassium, alitame, saccharin and its salts, cyclamic acid and its salts, glycyrrhizin, dihydrochalcones, thaumatin, monellin, stevioside, alone or in combination, are also usable within the scope of the invention.

In the above examples a peppermint flavor in powder form was used. A mixture of peppermint and menthol may also be used. Of course, other flavors as described herein may be used as well, in combination with menthol and/or peppermint or replacing these. The flavor may be liquid or powdered or a combination, i.e. a liquid flavor and a powdered flavor is added.

In the above sodium stearyl fumerate is used as lubricant. Other lubricants, such as MgSt (magnesium stearate) may also be usable within the scope of the invention.

Sodium carbonate is used as the alkaline pH regulating agent. Further usable alkaline pH regulating agents include sodium bicarbonate, potassium carbonate, potassium bicarbonate, trometamol, amino acids, disodium hydrogen phosphate, dipotassium hydrogen phosphate, trisodium phosphate, tripotassium phosphate, or any combination thereof.

### Example 3B

Tablets were prepared according to example 1, unless otherwise specified.

In this example, the following conditions were applied. Punch used: 7.00 mm, circular, shallow concave, B tooling. Tablet weight: 100.0 mg.

TABLE 3
Composition of tablets.

| | LFT | LFT | LFT | LFT | LFT | LFT | LFT |
|---|---|---|---|---|---|---|---|
| | (11) | (12) | (13) | (14) | (15) | (16) | (17) |
| Nicotine dose | 2.0 | 2.0 | 2.0 | 2.0 | 2.0 | 2.0 | 2.0 |
| [mg] | | | | | | | |
| Raw material | Content in weight percent | | | | | | |
| NBT | 6.2 | 6.2 | 6.2 | 6.2 | 6.2 | 6.2 | 6.2 |
| MCC | 2 | 2 | 2 | 2 | 2 | 2 | 2 |
| Maltitol | 82 | 79.5 | 74.5 | 72 | 77 | 77 | 79.9 |
| Crospovidone | 8 | 8 | 8 | 8 | 8 | 8 | 8 |
| Guar gum | 0.4 | 0.4 | 0.4 | 0.4 | 0.4 | 0.4 | - |
| Flavor | 0.2 | 0.2 | 0.2 | 0.2 | 0.2 | 0.2 | 0.2 |
| HIS | 0.2 | 0.2 | 0.2 | 0.2 | 0.2 | 0.2 | 0.2 |
| Sodium | — | 2.5 | 7.5 | 10 | — | 2.5 | 2.5 |
| carbonate | | | | | | | |
| Trometamol | — | — | — | — | 5 | 2.5 | — |
| Magnesium | 1 | 1 | 1 | 1 | 1 | 1 | 1 |
| stearate | | | | | | | |
| Total | 100 | 100 | 100 | 100 | 100 | 100 | 100 |

NBT = nicotine bi-tartrate (nicotine content of 32.38% by weight).
MCC = microcrystalline cellulose.
HIS = High intensity sweeteners.

Alternative ingredients as described in relation to LFT (1)-LFT (4) of table 2 may also be applied for LFT (11)-LFT (17).

### Example 3D

Tablets were prepared according to example 1, unless otherwise specified.

In this example, the following conditions were applied. Punch used: 7.00 mm, circular, shallow concave, B tooling. Tablet weight: 100.0 mg.

TABLE 4
Composition of tablets.

| | LFT(21) | LFT(22) | LFT(23) | LFT(24) |
|---|---|---|---|---|
| Nicotine dose [mg] | 2.0 | 2.0 | 2.0 | 2.0 |
| Raw material | Content in weight percent | | | |
| NBT | 6.2 | 6.2 | 6.2 | 6.2 |
| MCC | — | 10 | 20 | 40 |
| Mannitol | 78.8 | 68.8 | 58.8 | 38.8 |
| Crospovidone | 8 | 8 | 8 | 8 |
| Guar gum | 0.4 | 0.4 | 0.4 | 0.4 |
| Flavor | 0.3 | 0.3 | 0.3 | 0.3 |
| HIS | 0.3 | 0.3 | 0.3 | 0.3 |
| Sodium carbonate | 5 | 5 | 5 | 5 |
| MgSt | 1 | 1 | 1 | 1 |
| Total | 100 | 100 | 100 | 100 |

NBT = nicotine bi-tartrate (nicotine content of 32.38% by weight). MCC = microcrystalline cellulose.

Alternative ingredients as described in relation to LFT (1)-LFT (17) of tables 2-3 may also be applied for LFT (21)-LFT (24).

### Example 3E

Tablets were prepared according to example 1, unless otherwise specified.

In this example, the following conditions were applied. Punch used: 7.00 mm, circular, shallow concave, B tooling. Tablet weight: 100.0 mg.

TABLE 5
Composition of tablets.

| | LFT | LFT | LFT | LFT | LFT | LFT | LFT |
|---|---|---|---|---|---|---|---|
| | (31) | (32) | (33) | (34) | (35) | (36) | (37) |
| Nicotine dose [mg] | 2.0 | 2.0 | 2.0 | 2.0 | 2.0 | 2.0 | 2.0 |
| Raw material | Content in weight percent | | | | | | |
| NBT | 6.2 | 6.2 | 6.2 | 6.2 | 6.2 | 6.2 | 6.2 |
| MCC | 2 | 2 | 2 | 2 | 2 | 2 | 2 |
| Mannitol | 82.7 | 80.2 | 75.2 | 77.2 | 77.2 | 77.2 | 77.2 |
| Crospovidone | 2.5 | 5 | 10 | 4 | — | — | — |
| Croscarmellose | — | — | — | 4 | 8 | — | — |
| Sodium | | | | | | | |
| Sodium Starch | — | — | — | — | — | 8 | — |
| Glycolate | | | | | | | |
| Pregelatinized starch | — | — | — | — | — | — | 8 |
| Guar gum | 0.4 | 0.4 | 0.4 | 0.4 | 0.4 | 0.4 | 0.4 |
| Flavor | 0.1 | 0.1 | 0.1 | 0.1 | 0.1 | 0.1 | 0.1 |
| HIS | 0.1 | 0.1 | 0.1 | 0.1 | 0.1 | 0.1 | 0.1 |
| Sodium carbonate | 5 | 5 | 5 | 5 | 5 | 5 | 5 |
| Magnesium stearate | 1 | 1 | 1 | 1 | 1 | 1 | 1 |
| Total | 100 | 100 | 100 | 100 | 100 | 100 | 100 |

NBT = nicotine bi-tartrate (nicotine content of 32.38% by weight).
MCC = microcrystalline cellulose.
HIS = High intensity sweeteners.

Alternative ingredients as described in relation to LFT (1)-LFT (25) of tables 2-4 may also be applied for LFT (31)-LFT (36).

### Example 3F

Tablets were prepared according to example 1, unless otherwise specified.

In this example, the following conditions were applied. Punch used: 7.00 mm, circular, shallow concave, B tooling. Tablet weight: 100.0 mg.

TABLE 6
Composition of tablets.

| | LFT | LFT | LFT | LFT | LFT |
|---|---|---|---|---|---|
| | (41) | (42) | (43) | (44) | (45) |
| Nicotine dose [mg] | 1.0 | 3.0 | 1.0 | 2.0 | 2.0 |
| Raw material | Content in weight percent | | | | |
| NBT | 3.1 | 9.3 | 3.1 | — | — |
| NPR | — | — | 6.20 | 12.50 | — |
| Nicotine free base | — | — | — | — | 2.0 |
| MCC | 2 | 2 | 2 | 2 | 2 |
| Mannitol | 82.4 | 71.2 | 73.7 | 70.5 | 81 |
| Crospovidone | 8 | 8 | 8 | 8 | 8 |
| Guar gum | 0.4 | 0.4 | 0.4 | 0.4 | 0.4 |
| Flavor | 0.3 | 0.3 | 0.3 | 0.3 | 0.3 |
| HIS | 0.3 | 0.3 | 0.3 | 0.3 | 0.3 |
| Sodium carbonate | 2.5 | 7.5 | 5 | 5 | 5 |
| Magnesium stearate | 1 | 1 | 1 | 1 | 1 |
| Total | 100 | 100 | 100 | 100 | 100 |

NBT = nicotine bi-tartrate (nicotine content of 32.38% by weight). MCC = microcrystalline cellulose. NPR = nicotine polacrilex resin (nicotine content of 16% by weight). HIS = High intensity sweeteners.

Alternative ingredients as described in relation to LFT (1)-LFT (36) of tables 2-5 may also be applied for LFT (41)-LFT (45).

### Example 3G

Tablets were prepared according to example 1, unless otherwise specified.

In this example, the following conditions were applied. Punch used: 7.00 mm, circular, shallow concave, B tooling. Tablet weight: 100.0 mg.

TABLE 7
Composition of tablets.

| | LFT | LFT | LFT | LFT | LFT | | |
|---|---|---|---|---|---|---|---|
| | (51) | (52) | (53) | (54) | (55) | C(1) | C(2) |
| Nicotine dose [mg] | 2.0 | 2.0 | 2.0 | 2.0 | 2.0 | 1.0 | 2.0 |
| Raw material | Content in weight percent | | | | | | |
| NBT | 6.2 | 6.2 | 6.2 | 6.2 | 6.2 | 3.1 | 6.2 |
| MCC | 2 | 2 | 2 | 2 | 2 | 7.5 | 7.5 |
| Mannitol | 77.5 | 77.4 | 77.3 | 77.15 | 77.3 | 75.9 | — |
| Sorbitol | — | — | — | — | — | — | 72.8 |
| Crospovidone | 8 | 8 | 8 | 8 | 8 | 8 | 8 |
| Menthol flavor | — | 0.1 | 0.2 | 0.2 | — | 2 | 2 |
| Tobacco flavor | — | — | — | 0.15 | 0.2 | — | — |
| HIS | 0.3 | 0.3 | 0.3 | 0.3 | 0.3 | 0.5 | 0.5 |
| Sodium carbonate | 5 | 5 | 5 | 5 | 5 | 2 | 2 |
| Magnesium stearate | 1 | 1 | 1 | 1 | 1 | — | — |
| Sodium stearyl | — | — | — | — | — | 1 | 1 |
| fumarate | | | | | | | |
| Total | 100 | 100 | 100 | 100 | 100 | 100 | 100 |

NBT = nicotine bi-tartrate (nicotine content of 32.38% by weight).
MCC = microcrystalline cellulose.
HIS = High intensity sweeteners.

Alternative ingredients as described in relation to LFT (1)-LFT (45) of tables 2-5 may also be applied for LFT (51)-LFT (55).

### Example 3H

Tablets were prepared according to example 1, unless otherwise specified.

In this example, the following conditions were applied. Punch used: 7.00 mm, circular, shallow concave, B tooling. Tablet weight: 100.0 mg.

TABLE 8
Composition of tablets.

| | LFT | LFT | LFT | LFT | LFT | LFT | LFT |
|---|---|---|---|---|---|---|---|
| | (61) | (62) | (63) | (64) | (65) | (66) | (67) |
| Nicotine dose [mg] | 2.0 | 2.0 | 2.0 | 2.0 | 2.0 | 2.0 | 2.0 |
| Raw material | Content in weight percent | | | | | | |
| NBT | 6.2 | 6.2 | 6.2 | 6.2 | 6.2 | 6.2 | 6.2 |
| MCC | 2 | 2 | 2 | 2 | 2 | 2 | 2 |
| Mannitol | 82.8 | 80.3 | 75.3 | 77.3 | 77.3 | 77.3 | 77.3 |
| Crospovidone | 2.5 | 5 | 10 | 4 | — | — | — |
| Croscarmellose | — | — | — | 4 | 8 | — | — |
| Sodium | | | | | | | |
| Sodium Starch | — | — | — | — | — | 8 | — |
| Glycolate | | | | | | | |
| Pregelatinized starch | — | — | — | — | — | — | 8 |
| Guar gum | 0.4 | 0.4 | 0.4 | 0.4 | 0.4 | 0.4 | 0.4 |
| Flavor | 0.1 | 0.1 | 0.1 | 0.1 | 0.1 | 0.1 | 0.1 |
| Sodium carbonate | 5 | 5 | 5 | 5 | 5 | 5 | 5 |
| Magnesium stearate | 1 | 1 | 1 | 1 | 1 | 1 | 1 |
| Total | 100 | 100 | 100 | 100 | 100 | 100 | 100 |

NBT = nicotine bi-tartrate (nicotine content of 32.38% by weight).
MCC = microcrystalline cellulose.
HIS = High intensity sweeteners.

Alternative ingredients as described in relation to LFT (1)-LFT (55) of tables 2-7 may also be applied for LFT (61)-LFT (67).

### Example 4: Tablet Compositions, Two Layer Tablets

### Example 4A

Tablets were prepared according to example 2, unless otherwise specified.

In this example, the following conditions were applied. Punch used: 7.00 mm, circular, shallow concave, B tooling. Weight of 1^{st} layer: 100.0 mg. Weight of 2^{nd} layer: 300.0 mg. Tablet weight: 400.0 mg

TABLE 9
Composition of tablets.

| | LFT | LFT | LFT | LFT | LFT |
|---|---|---|---|---|---|
| | (71) | (72) | (73) | (74) | (75) |
| Nicotine dose 1^{st} | 1.0 | 3.0 | 1.0 | 2.0 | 2.0 |
| layer [mg] | | | | | |
| Raw material 1^{st} | Content in weight percent of 1^{st} layer | | | | |
| layer | | | | | |
| NBT | 3.1 | 9.3 | 3.1 | 6.2 | — |
| Nicotine free base | — | — | — | — | 2.0 |
| MCC | 2.4 | 2.4 | 2.4 | 2.4 | 2.4 |
| Mannitol | 82.4 | 71.2 | 79.9 | 76.8 | 81 |
| Crospovidone | 8 | 8 | 8 | 8 | 8 |
| Flavor | 0.3 | 0.3 | 0.3 | 0.3 | 0.3 |
| HIS | 0.3 | 0.3 | 0.3 | 0.3 | 0.3 |
| Sodium carbonate | 2.5 | 7.5 | 5 | 5 | 5 |
| Magnesium | 1 | 1 | 1 | 1 | 1 |
| stearate | | | | | |
| Total 1^{st} layer | 100 | 100 | 100 | 100 | 100 |
| Nicotine dose 2^{nd} | 3.0 | 1.0 | 3.0 | 3.0 | — |
| layer [mg] | | | | | |
| Raw material 2nd | Content in weight percent of 2^{nd} layer | | | | |
| layer | | | | | |
| NBT | 3.1 | 1 | 1 | — | — |
| NPR | — | — | 4.2 | 6.3 | — |
| Mannitol | 91.2 | 93.1 | — | — | 98.9 |
| Xylitol | — | — | 88.7 | 87.9 | — |
| Flavor | 0.1 | 0.2 | 0.3 | 0.3 | 0.3 |
| HIS | 0.1 | 0.2 | 0.3 | — | 0.3 |
| Sodium carbonate | 5 | 5 | 5 | 5 | — |
| Magnesium | 0.5 | 0.5 | 0.5 | 0.5 | 0.5 |
| stearate | | | | | |
| Total 2^{nd} layer | 100 | 100 | 100 | 100 | 100 |

NBT = nicotine bi-tartrate (nicotine content of 32.38% by weight). MCC = microcrystalline cellulose. NPR = nicotine polacrilex resin (nicotine content of 16% by weight). HIS = High intensity sweeteners.

Alternative ingredients as described in relation to LFT (1)-LFT (67) of tables 2-8 may also be applied for LFT (71)-LFT (75).

### Example 4B

Further tablets were made using the tablet of any of LFT (1)-LFT (67) of tables 2-8 as the first layer and the second layer of one of LFT (71)-LFT (75).

### Example 5: Evaluation

The produced tablets of the invention were evaluated and found highly suitable as delivery vehicles of nicotine in that they provide a favorable release of nicotine and at the same time are pleasant to the user, e.g. with respect to an acceptable nicotine burning while leading to a not too high flavor perception and aftertaste.

### Example 6: Evaluation

Evaluation of comparative orally disintegrating nicotine tablets C(1) and C(2) was made by a number of assessors. The number of assessors was 84 for C(1) and 94 for C(2).

For C(1) and C(2), the assessors were asked to indicate if the flavor intensity during use was too strong, or too weak. Below table 10 shows the ratios between the number of assessors indicating a too strong intensity of the taste compared to a too weak intensity of the taste. Also, the duration of taste was evaluated and the ratio between the number of assessors indicating a too long duration of the taste compared to a too short duration of the taste.

TABLE 10
Evaluation of taste.

| | Flavor intensity during use | Duration of taste |
|---|---|---|
| | Ratio between too strong and | Ratio between too long and |
| | too weak | too short |
| C(1) | 3.3 | N/A |
| C(2) | 1.9 | 3.0 |

N/A = not evaluated.

As seen from table 10, an unexpectedly high fraction of assessors indicated that the flavor intensity was too high. Similarly, an unexpectedly high fraction of the assessors indicated the taste to be lasting too long.

Since flavor typically is included to mask the taste of nicotine, it was quite unexpected that a relatively high fraction of assessors found the flavor intensity to be too high, compared to the opposite.

Similarly, both comparative tablets were assessed with respect to aftertaste, where the ratios was observed between the number of assessors indicating a too strong intensity of the aftertaste compared to a too weak intensity of the aftertaste. Also, the duration of aftertaste was evaluated as the ratio between the number of assessors indicating a too long duration of the aftertaste compared to a too short duration of the aftertaste.

TABLE 11
Evaluation of aftertaste.

| | Intensity of aftertaste | Duration of aftertaste |
|---|---|---|
| | Ratio between too strong and | Ratio between too long and |
| | too weak | too short |
| C(1) | 4.6 | 6.0 |
| C(2) | 6.3 | 3.8 |

As seen from table 11, the evaluation of aftertaste showed an unexpected preference for a shorter and less intense aftertaste.

The above unexpected findings of tables 10-11 show that a significant segment of assessors had a preference for a low amount or even absence of flavor, as provided by the inventive orally disintegrating nicotine tablets disclosed herein.

1. An orally disintegrating nicotine tablet for fast nicotine craving relief,
the tablet comprising nicotine, disintegrant, and at least one sugar alcohol,
wherein the tablet comprises less than 0.4% by weight of flavor.

2. The orally disintegrating nicotine tablet according to claim 1, wherein the tablet comprises no more than 0.3% by weight of flavor.

3. (canceled)

4. The orally disintegrating nicotine tablet according to claim 1, wherein the tablet comprises less than 0.4% by weight of liquid flavor.

5. (canceled)

6. The orally disintegrating nicotine tablet according to claim 1, wherein the tablet comprises less than 0.4% by weight of powdered flavor.

7. The orally disintegrating nicotine tablet according to claim 1, wherein the tablet is free of flavor.

8. The orally disintegrating nicotine tablet according to claim 1, wherein the tablet comprises less than 0.4% by weight of high intensity sweetener.

9. The orally disintegrating nicotine tablet according to claim 1, wherein the tablet is free of high intensity sweetener.

10. (canceled)

11. The orally disintegrating nicotine tablet according to claim 1, wherein the tablet comprises the at least one sugar alcohol sugar alcohol in an amount of at least 40% by weight of the tablet.

12. The orally disintegrating nicotine tablet according to claim 1, wherein the at least one sugar alcohol comprises sugar alcohol selected from the group consisting of xylitol, maltitol, mannitol, erythritol, isomalt, sorbitol, lactitol and any combination thereof.

13. The orally disintegrating nicotine tablet according to claim 1, wherein the tablet comprises said disintegrant in an amount of at least 0.5% by weight of the tablet.

14. The orally disintegrating nicotine tablet according to claim 1, wherein said disintegrant is selected from the list consisting of starch, pregelatinated starch, modified cellulose, ion-exchange resin, calcium silicate, crosslinked cellulose, crosslinked polyvinyl pyrrolidone, crosslinked starch, crosslinked alginate, and combinations thereof.

15-16. (canceled)

17. The orally disintegrating nicotine tablet according to claim 1, wherein the disintegrant comprises a superdisintegrant.

18. The orally disintegrating nicotine tablet according to claim 1, wherein the tablet comprises superdisintegrant in an amount of at least 0.5% by weight of the tablet.

19. The orally disintegrating nicotine tablet according to claim 17, wherein the superdisintegrant is selected from the group consisting of crosslinked cellulose, crosslinked polyvinyl pyrrolidone, crosslinked starch, crosslinked alginate, and any combinations thereof.

20-21. (canceled)

22. The orally disintegrating nicotine tablet according to claim 1, wherein the tablet is a compressed tablet.

23-24. (canceled)

25. The orally disintegrating nicotine tablet according to claim 1, wherein the nicotine is selected from the group consisting of a nicotine salt, nicotine free base, a nicotine-ion exchange resin combination, a nicotine inclusion complex or nicotine in any non-covalent binding, nicotine bound to zeolites, nicotine bound to cellulose, nicotine bound to starch microspheres, and any combination thereof.

26-29. (canceled)

30. The orally disintegrating nicotine tablet according to claim 1, wherein the tablet comprises nicotine in an amount of at least 0.2 mg.

31. (canceled)

32. The orally disintegrating nicotine tablet according to claim 1, wherein the tablet comprises a pH regulating agent.

33. (canceled)

34. The orally disintegrating nicotine tablet according to claim 32, wherein the pH regulating agent comprises pH regulating agent selected from the group consisting of sodium carbonate, sodium bicarbonate, potassium carbonate, potassium bicarbonate, trometamol, amino acids, di-alkali hydrogen phosphate, tri-alkali phosphate, or any combination thereof.

35-36. (canceled)

37. The orally disintegrating nicotine tablet according to claim 32, wherein the tablet comprises the pH regulating agent in an amount of at least 0.2% by weight of the tablet.

38-42. (canceled)

43. The orally disintegrating nicotine tablet according to claim 1, wherein the tablet further comprises dissolution modifiers in an amount of at most 2% by weight of the tablet.

44-49. (canceled)

50. An orally disintegrating nicotine tablet for fast nicotine craving relief,
the tablet comprising nicotine, disintegrant, and at least one sugar alcohol,
wherein the tablet has a total amount of flavor and high intensity sweetener of less than 0.8% by weight of the tablet.

51-52. (canceled)

53. An oral nicotine tablet comprising the orally disintegrating nicotine tablet according to claim 1 as a first module and further comprising a second module,
wherein the second module is different in composition from the first module.

54-63. (canceled)
